(19) United States
(12) Patent Application Publication
(10) Pub. No.: US 2009/0240334 A1
(43) Pub. Date: Sep. 24, 2009

(54) VERTEBRAL DEVICE FOR RESTORATION OF VERTEBRAL BODY HEIGHT

(76) Inventor: Marc E. Richelsoph, Belmont, NC (US)

Correspondence Address: KOHN & ASSOCIATES, PLLC, 30500 NORTHWESTERN HWY, SUITE 410, FARMINGTON HILLS, MI, 48334, US

(21) Appl. No.: 12/051,491

(22) Filed: Mar. 19, 2008

(51) Int. Cl. A61F 2/44

(52) U.S. Cl. 623/17.16

(57) ABSTRACT

An intra-vertebral body height restoring device includes a body for insertion into an intra-vertebral space. The body includes top and bottom surfaces for engaging opposing vertebral surfaces defining the intra-vertebral space. The body includes at least two layers extending along a width of the body and having a fully expanded and fully collapsed height relative thereto. A reversible expansion mechanism selectively and reversely expands and collapse the height of the layers and including the fully expanded and collapsed heights to restore a selected height to the intra-vertebral space.

### BACKGROUND OF THE INVENTION

1. Field of the Invention

The present invention generally relates to a kyphoplasty device. More specifically, the present invention relates to a vertebral body height restoration device which assists in restoring the loss of height of a vertebral body by forcing apart opposing vertebral end plates.

2. Description of Related Prior Art

Kyphoplasty and vertebroplasty procedures have been in use for many years. Percutaneous vertebroplasty involves injecting bone cement into a weakened or damaged vertebral body in an attempt to relieve pain and stabilize a collapsed vertebral body. The procedure is performed utilizing a needle under fluoroscopy as a percutaneous approach. Kyphoplasty is a more recently developed procedure whereby the vertebral fracture is reduced by utilizing a bone tamp with an inflatable balloon to create a cavity for bone cement and eventually force the vertebral end plates apart to restore vertebral body height.

Typically, kyphoplasty devices include a balloon contained within a cannula. The balloon is inflated after introduction into the damaged vertebral body. Under fluoroscopy, the balloon can be inflated to exert force to assist in restoring height. Once this step is completed, the balloon is deflated, removed, and bone cement is injected into the cavity. The balloons are simple inflatable elastomeric containers that are inflated into a rounded or oval shape.

There are significant problems with the aforementioned approaches. First, an inflatable balloon includes a radius such that the top point of the radius creates a very limited pressure applying area for applying pressure against the vertebral end plates and separating the end plates as a result of this applied pressure. This limits the accuracy of height and lordotic restoration. Secondly, the cavity created for the bone cement usually duplicates the shape of the balloon. This rounded shape does not create the best means for stabilizing the adjacent end plates. In addition, the bone cement is injected into a compromised vertebral body which usually includes fractures which are open to the body. Thus, it is possible for bone cement to be forced by the pressure applied outside of the vertebral body and into areas surrounding the spine. The results of such are disastrous and potentially lethal.

While the aforementioned devices may be suitable for the particular purpose to which they address, they are not as suitable for providing a device that provides accurate restoration of vertebral body height and lordotic angle. Furthermore, the prior art procedures and devices do not allow for containment of the bone cement during the bone cement injection procedure.

In view of the above, the present invention substantially departs from the conventional concepts and designs of the prior art and in doing so, provides an apparatus primarily developed for the purpose of accurately restoring a vertebral body and spine dynamic while providing a means to contain the bone cement within the vertebral body during the bone cement injection procedure.

### SUMMARY OF THE INVENTION

In accordance with the present invention, there is provided an intra-vertebral body height restoring device including a body for insertion into an intra-vertebral space. The body includes top and bottom surfaces for engaging opposing vertebral surfaces defining the intra-vertebral space. The body further includes at least two layers extending along a width of the body and having a fully expanded and fully collapsed height relative thereto. A reversible expansion mechanism selectively and reversibly expands and collapses the height of the layers between and including the fully expanded and collapsed heights to restore a selected height of the intra-vertebral space.

The present invention further provides an intra-vertebral body height restoring device including a body defining a width and height and including an inner portion defining at least two layers extending along a width of the body and an expansion mechanism for selectively and reversibly expanding and collapsing the height of the layers.

The present invention also provides an intra-vertebral body height restoring device including a body and a reversible expansion mechanism for selectively and reversibly expanding and collapsing the body and a containment mechanism within the body for containing a hardenable fluid therein.

The present invention also provides an intra-vertebral body height restoring device including a body and a containment mechanism within the body for containing a hardenable fluid therein. A porous surface allows a selective amount of flow of the hardenable fluid from the contained amount of hardenable fluid within the body through at least one surface of the body for contact with a vertebral surface adjacent to the body surface.

In addition to the above, the present invention provides a method of restoring height to a collapsed intra-vertebral space by inserting a body into the intra-vertebral space defined by opposing vertebral surfaces and selectively and reversibly expanding layers of the body causing top and bottom surfaces of the body to contact and separate the opposing vertebral surfaces thereby expanding the intra-vertebral space.

A method is further provided for restoring height to a collapsed intra-vertebral space by expanding a body disposed within the intra-vertebral space to separate opposing vertebral surfaces defining the space and injecting bone cement into the expanded body while containing the bone cement within the body.

The present invention also provides a method of restoring height to a collapsed intra-vertebral space by injecting a hardenable material into layers of a body, expanding the height of the body with the hardenable material to separate adjacent vertebral surfaces defining the intra-vertebral space, and hardening the hardenable material to fixedly space the vertebral surfaces.

The present invention further provides a method of restoring height to an intra-vertebral space by expanding a body containing a hardenable material within the intra-vertebral space to separate opposing vertebral surfaces defining the space and selectively leaking the hardenable material through permeable top and bottom surfaces of the body to contact the hardenable fluid with selected portions of the adjacent vertebral surfaces.

Additionally, the present invention provides a device for restoring height of a collapsed intra-vertebral space, the device including an expandable body and programmable control mechanism for controlling expansion of the body to a predetermined height in view of a predetermined height.

### BRIEF DESCRIPTION OF THE DRAWINGS

Other advantages of the present invention are readily appreciated as the same becomes better understood by reference to the following detailed description when considered in connection with the accompanying drawings wherein:

FIG. 1 is a perspective view of the present invention;

FIG. 2 is a perspective view of the invention as shown in FIG. 1 rotated 90°;

FIG. 3 is a perspective view of the present invention showing a hollow core in transparent form;

FIG. 4 is a side view of the present invention showing a manifold and port arrangement for one embodiment of the present invention;

FIG. 5 is a review view of the present invention showing the manifold and port arrangement;

FIG. 6 is a perspective rear view of the present invention showing the manifold and port arrangement including a cannula for insertion;

FIG. 7 is a perspective view showing the hollow core of the body member of the present invention with an upper and lower seal barrier;

FIG. 8 is a perspective transparent view showing the hollow core of the present invention including an upper and lower seal barrier;

FIG. 9 is a cross-sectional view showing the hollow core with an upper and lower seal barrier as well as filling holes into the hollow core and cavity of the body portion;

FIG. 10 is a cross-sectional view showing the hollow core without the upper and lower seal barriers;

FIG. 11 is an enlarged transparent view of the hollow core device showing inner details including communication openings between layers;

FIG. 12 is a side view of the present invention where a top layer includes an angled surface;

FIG. 13 is a side view of the present invention including an angle top layer and a cannula disposed about the filling tube;

FIG. 14 is a perspective view of a solid core body made in accordance with the present invention;

FIG. 15 is a perspective transparent view of a solid core implant;

FIG. 16 is a transparent side view of the solid core implant with a top layer angled surface;

FIG. 17 is a perspective view of a hollow core cannula system;

FIG. 18 is a shaded transparent perspective view of a solid core implant;

FIG. 19 is a side view which is shaded and transparent of the solid core implant;

FIG. 20 is an enlarged shaded transparent side view of the solid core implant with an angled top surface;

FIG. 21 is a rear perspective view, which is transparent and shaded, of the hollow core implant;

FIG. 22 is a shaded transparent top perspective view of the hollow core implant showing interior detail;

FIG. 23 is a shaded transparent side perspective view of the hollow core implant including a cannula;

FIG. 24 is a top perspective view, shaded and transparent, of the hollow core implant including a cannula and showing interior detail;

FIG. 25 is an enlarged side perspective view, transparent and shaded, of the present invention;

FIG. 26 is a side perspective view of the body portion comprising a helical layered construction; and

FIG. 27 is a pneumatic diagram of an automated control system for feeding fluid to the present invention.

### DETAILED DESCRIPTION OF THE INVENTION

An intra-vertebral body height restoring device made in accordance with the present invention is generally shown at 10 in the figures. Most generally, the present invention includes a body 1 for insertion into an intra-vertebral space (not shown). The body 1 includes top and bottom surfaces 100, 102 for engaging opposing vertebral surfaces defining the intra-vertebral space. That is, the device 10 is to be inserted into an intra-vertebral space between two vertebrae. The two adjacent vertebrae include opposing vertebral surfaces that define the inter-vertebral space. It is this space, in a collapsed or otherwise damaged condition that is going to be expanded thus restoring height to the space and the final outcome of which the vertebrae are comprised.

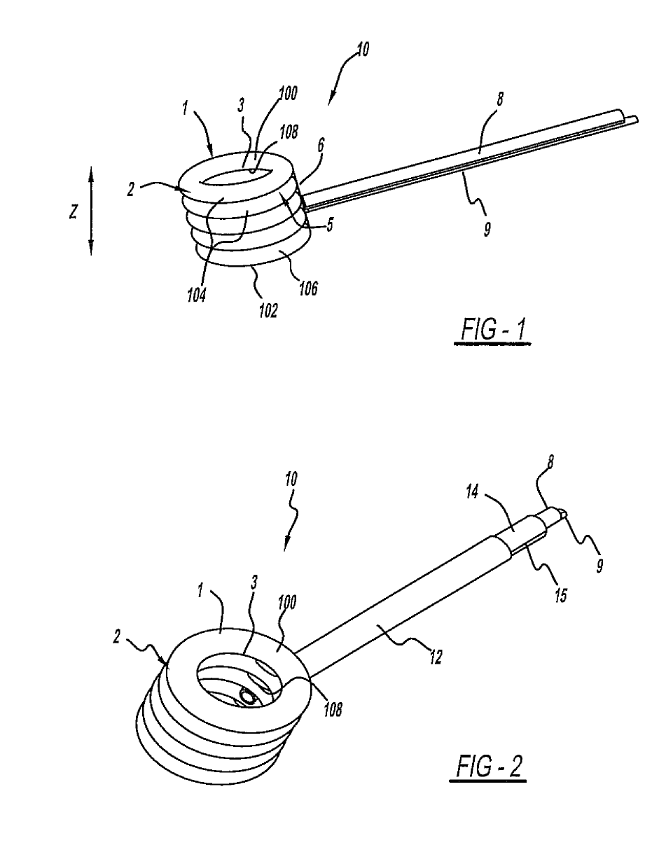

The body 1 includes at least two layers 104 extending along a width of the body 1, each of the layers 104 having a fully expanded and fully collapsed height relative thereto. A reversible expansion mechanism generally shown at 9 selectively and reversibly expands and collapses the height of the layers, the height being shown by arrow Z, between and including the fully expanded and collapsed heights to restore a selected height of the intra-vertebral space. That is, each of the layers 104 can selectively or collectively expand or collapse to increase the height in the Z direction as shown in FIG. 1 or decrease the height. Hence, the assembly can be inserted into an intra-vertebral space in the collapsed condition and then the body 1 is expanded to force the adjacent vertebrae apart as the top and bottom surfaces 100, 102 of the body 1 contact and force the opposing vertebral surfaces apart.

More specifically, and again referring to FIG. 1, the body 1 includes a radially outer peripheral surface 2 and each of the layers 104 include an inner surface 3, an upper surface 41 and a lower surface 5. These layers are effectively toroids or donuts having a ring configuration. In the various figures, the outer peripheral surface 2 defines a wall shown with a round cross section. However, the body 1 can take on various other shapes, such as an elliptical, square, or other shape. In the preferred embodiment, round sections are preferred as the shape is strongest for this application.

As stated above, in FIG. 1, five ring-shaped layers 104 are stacked, such that all of the layers or rings 104 are directly connected to each other. The number of layers or rings 104 is based on the height of the desired distraction, height of each layer in the final expanded shape, and wall thickness of each of the layers or rings. Each of these dimensions can be varied dependent on the needed use. Additionally, wall thickness, dimension, and expanded height can be varied depending on the required strength of the body 1 in order to contain a fluid or other means forcing the expansion of each of the layers 104. In other words, dimensions, wall thickness, etc., can be varied to prevent bursting of the system, depending on the forces required to increase the height of the intra-vertebral space by forcing apart the opposing vertebrae.

Still referring to FIG. 1, the lowermost ring, specifically labeled 106, includes the bottom surface 102 that in operation pushes against and applies a force to the vertebral end plate, also referred to above as one of the opposing vertebral surfaces. Alternatively, the bottom surface 102 can apply a force against cancellous bone. The exposed top surface 100 of the topmost ring 104 pushes against and applies an upward force as the layers 104 are expanded to restore the fractured or collapsed vertebrae back to its proper predetermined height.

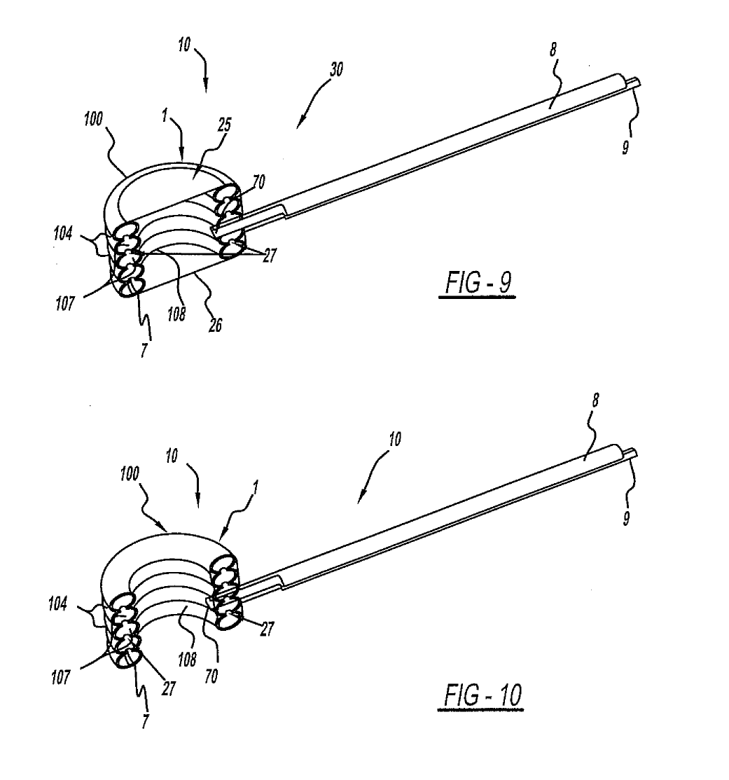

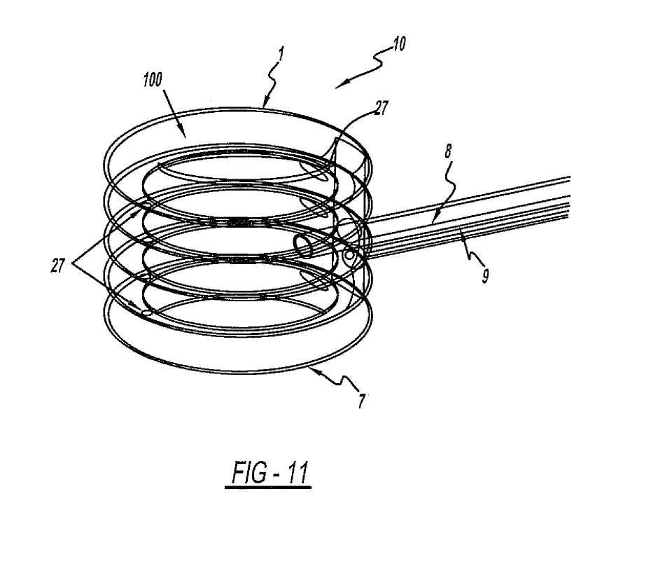

As shown in the various transparent views and in the various cross sectional views, such as FIGS. 9 and 10, in the preferred embodiment, each of the layers 104 includes a hollow inner chamber 107. A small tube 9 provides a fluid inlet mechanism for selectively and reversibly supplying a fluid to the inner chambers 107 of each of the layers to expand or collapse the height of the body 1. Fluids, such as sterile saline, or gases, such as air, can be delivered to the inner chambers 107 via the tube 9. Alternatively, various other means well known in the art for expanding or collapsing, or inflating or deflating an expandable chamber can be used. Various chemical and other mechanical means can be used consistent with the present invention.

Once the device 10 establishes the predetermined desired vertebral body height, bone cement or another hardenable fluid material, such as a bioactive bone substitute or bioresorbable bone cement is injected into the hollow core center of the device 10 to fill the space 108 defined within the inner wall 3. In other words, the inner wall 3 defines an open space therein for receiving a hardenable fluid therein. The space is shown as being cylindrical in form but can take on other shapes that may be needed in particular surgical situations.

The hardenable fluid material is injected into the hollow core 108 through tube 8. Thusly, tube 8 provides a second fluid inlet in fluid communication with the hollow inner core 108.

As show in the various Figures, tubes 8 and 9 are shown as separate tubes. However, as those skilled in the art would know, modern molding techniques can be used to mold a tube within a tube or even multiple smaller tubes within a larger tube. In other words, various tube configurations can be utilized to accomplish the dual filling functions of tubes 8 and 9. For example, an essentially single tube structure is shown in FIG. 2. The single tube has dual filling attachments to reduce the overall size of the insertion cannula 12.

For insertion of the device 10, the device 10 is contained and protected within a cannula 12. Cannula 12 is shown in various of the drawings, such as FIGS. 2, 6, and 7. During the insertion process, the device 10 is contained and protected within the cannula 12. The device 10 is then pushed out of the cannula 12 by sliding the cannula over an internal guide shown at 14 in FIGS. 2, 6, and 7. The cannula 12 can be keyed to the internal guide by way of a flat or keyway 15 to guarantee that the device 10 is aligned in the proper direction prior to introduction of the fluid into the various layers 104 for enlarging the body 1 within the intra-vertebral space.

It is critical that the body 1 be aligned so that the top and bottom surfaces 100, 102 are adjacent to and in eventual contact with the opposing vertebral surfaces defining the intra-vertebral space. These alignment means in the form of the internal guide 14 and the fiat keyway 15 give the practitioner assurance of this desired alignment.

Once the hardenable material is injected into the hollow core 108 of the device 101 it is allowed to harden. Once it is hard enough to support the load placed by the surrounding vertebrae, fluid or gas used to enlarge the device 10 can be vented. In other words, the fluid inlet 8 allows for injection of and venting of the gas or fluid used to enlarge the layers 104 of the device 10. It is possible to use the device 10, which is in the form of an implant, to support the vertebral end plates during the healing process by leaving the device 10 in the expanded condition. This allows the implant to share the load with the bioresorbable material used to fill the middle hollow core of the implant. However, when this is done, the loads on the implant require different design considerations than if the implant is only used to temporarily support the load. Alternatively, the layers 104 of the body 1 can be constructed from a bioresorbable flexible polymer or material so that the device is only present for the time that it is needed. Absorption of the material can be controlled by the chemical nature of the material to coordinate the resorption with the projected time of healing.

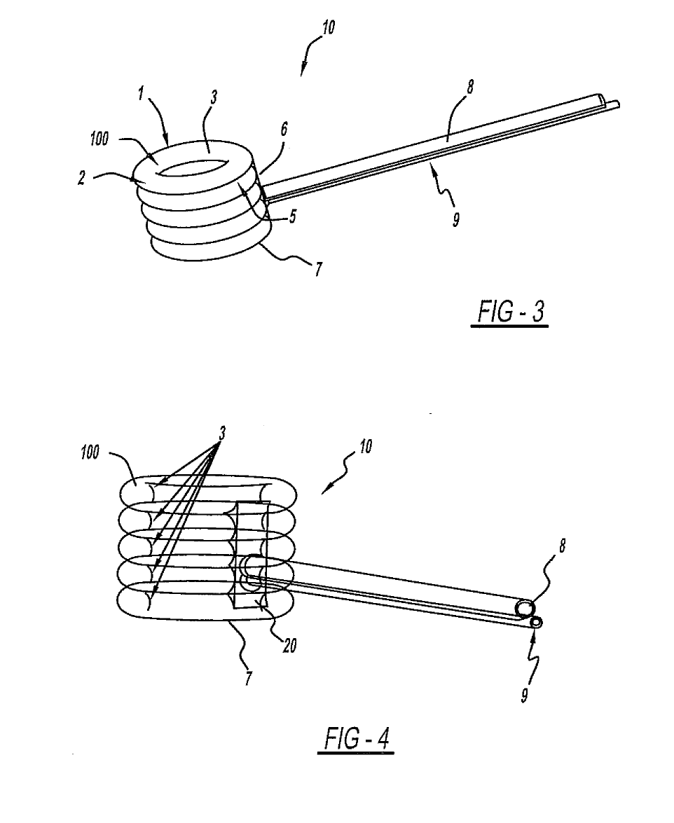

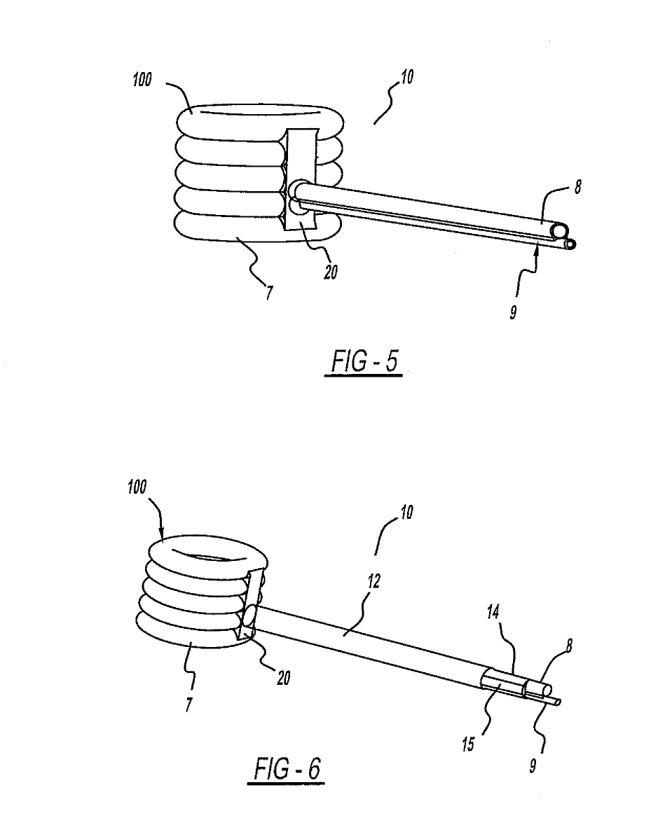

As shown in FIGS. 1-6, the hollow core 108 of the body 1 is completely open through the middle of the body 1 to allow bone cement or other hardenable filler material or fluid to exit only at the opening in the upper surface 100 and lower surface 102. This allows the filler material to integrate and interdigitate with the upper and lower end plates and cancellous bone while minimizing or preventing bone cement from leaking out the sides of the vertebral body. When used in the case of a fractured or a severely collapsed vertebral body, upon restoration of the height thereof the fracture is now open. By using this hollow core device 10 of this embodiment, the tubular external sidewalls of the body 10 act as a barrier to leakage. Accordingly, the device provides a much safer use of bone cement and helps to restrict it to where the surgeon desires it to be.

In accordance with the method of using the inventive device 10, it is also important to note that as the bone cement or hardenable material is injected into the hollow center core 108, as the material increases in quantity and/or pressure, the fluid or gas used to expand the layers 104 of the body 1 can be vented out of the device 10 to allow maximum fill of the vertebral body. This can be done manually or through a control valve. Alternatively, this can be done though an automated system as discussed below.

As shown in FIGS. 4-6, a reinforcement 20 operatively connected to various layers 104 allows an effective web of increased material for stronger attachment of the fluid/gas tube 9 and the hardenable fluid/bone cement tube 8. The reinforcement 20 specifically securely connects fluid/gas tube 9 in fluid communication with the inner chambers of layers 104 while also securely connecting the bone cement tube 8 through the walls of the body 1 then into the hollow inner core 108. This reinforcement section also acts as a manifold from layer to layer of the body 1 to allow the fluid or gas to fill each chamber within each layer 104 without entering the bone cement tube 8. Of course, there are other methods of molding the device and other approaches as shown in other figures.

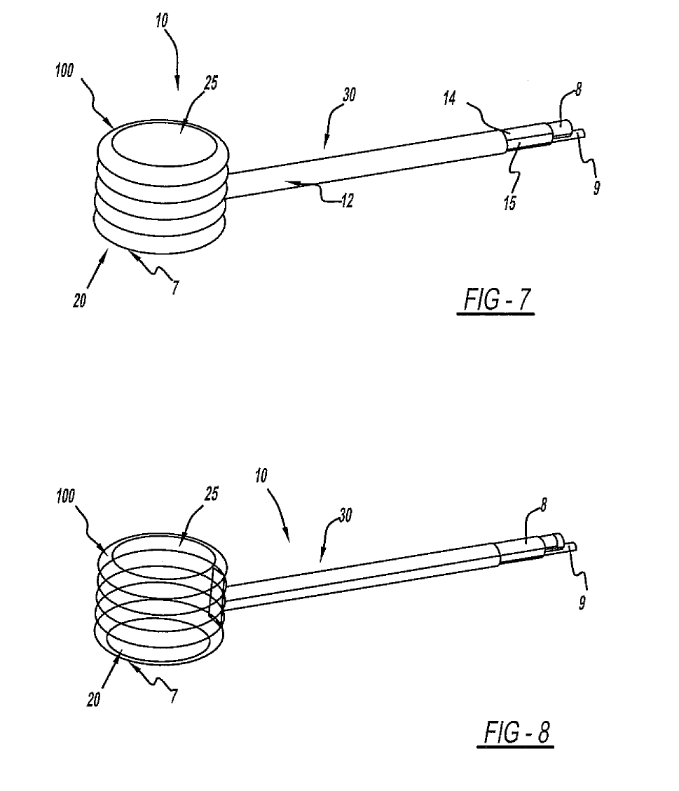

FIGS. 7-9 show a variation in the structure of the body member, this embodiment being generally shown at 30. In this embodiment, the hollow central core 108 is still in fluid communication with the inlet tube 8, however, end caps 25 and 26 seal the upper and lower rings. These flexible thin wall caps 25, 26 seal the hollow inner core 108 such that a hollow cavity is created with no passage therefrom, except through the injection tube 8. Thus, when the hardenable material or cement or other material is injected through the tube 8, the hardenable material cannot leak outside of the device 10. The hardenable material becomes trapped in the central core of the body 1. As best shown in FIGS. 9 and 10, the tip 70 of the inlet tube 8 is open to the center of the open chamber 108. For severe fractures, this embodiment has significant advantages, as the material injected into the hollow core 108 is trapped therein.

In a further embodiment on this approach, the end caps 25 and 26 are made from a porous or semi-porous material. Accordingly, the end caps 25, 26, limit the amount of bone cement or alternative that can leak therethrough to engage the end plates as the hardenable material leaks out of the implant. In fractures or when low viscosity injectible materials are used, this controlled and selective release of the hardenable fluid assures the maintenance of the hardenable fluid within the vertebral body. Of course, various porous materials and materials having various pore sizes and permeability can be used depending on the materials being injected and the desired amount of leakage desired.

FIG. 9 shows a cross-sectional view of the body 1, demonstrating the fluid gas passages 27 between the inner chambers of the layers 104. In this manner, a single fluid inlet 9 can be used to expand or collapse all of the various chambers 106. These openings 27 can be in various shapes and vary in number and size consistent with the present invention.

FIG. 10 is a cross-sectional view of the body 1 without end caps 25, 26 also showing the fluid gas passageways 27 that allow for fluid communication between the individual chambers 106. Again, these openings 27 between the chambers 106 can be of any shape and vary in number and location. FIG. 7 is an enlarged view showing the structural features.

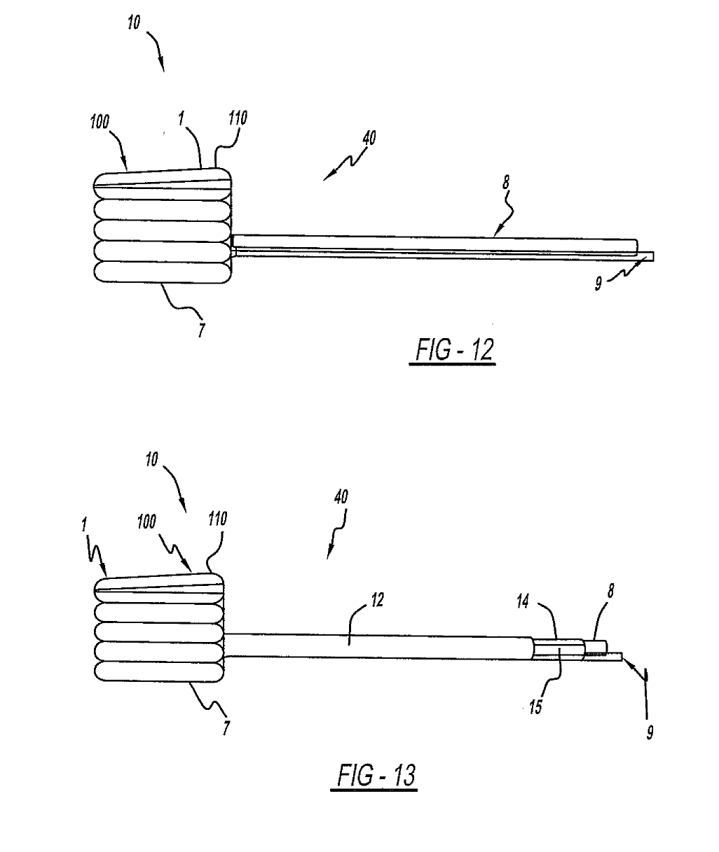

FIGS. 12 and 13 show the device 10 including the body 1 having the hollow core therein with an angled face 110 on the uppermost of the layers, which becomes a device generally described in the embodiment 40. By using an angled face 110, the present invention can be shaped to better match the angle of the vertical end plates to assist in restoring the proper lordosis to the spine. The device provides a mechanism for restoring proper lordosis. If the device is rotated 180° such that the angle of the face is in the opposite direction, while still in the highermost layer, the higher end of the angled face touches the more anterior aspect of the end plate or cancellous bone. This configuration provides a higher relative pressure interiorly to force apart the end plates and can be used in severe vertebral body collapse situations.

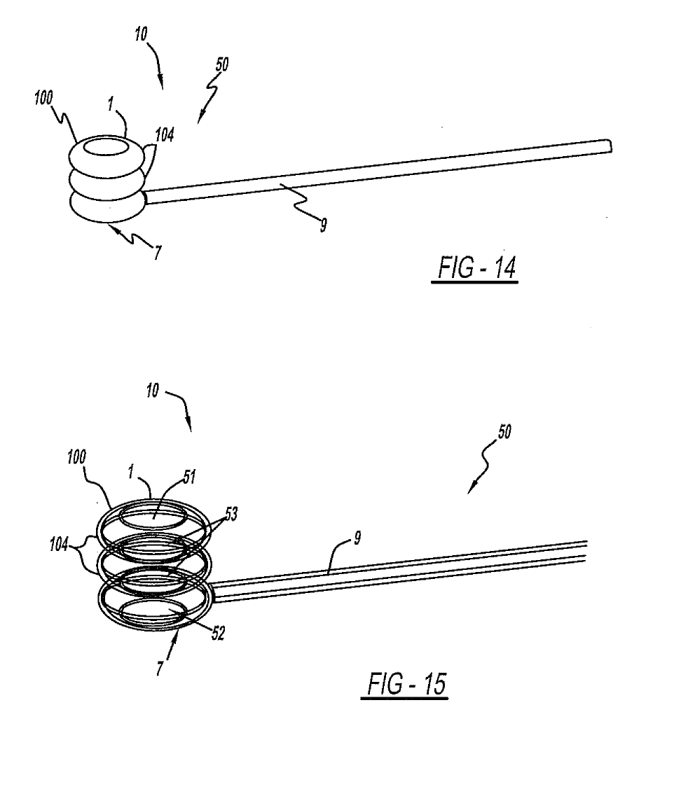

FIGS. 14 and 15 show a further embodiment of the present invention generally shown at 50. This embodiment 50 provides a solid core device. The solid core is provided by the device 50 not having an open hollow core therein or channel for the introduction of bone cement or other materials into a hollow core. Rather, the hardenable fluid is injected directly into the inner chambers of the layers 104 of the body 1. Therefore, the device 50 is a closed system designed to provide an instrument that can restore the vertebral body height and geometry while creating a cavity inside the intra-vertebral space for the introduction of a hardening material.

The device is inserted into the intra-vertebral space and expanded to the desired height. The device is then removed from the space and bone cement or other suitable material is injected into the cavity created by the expansion of the device 50.

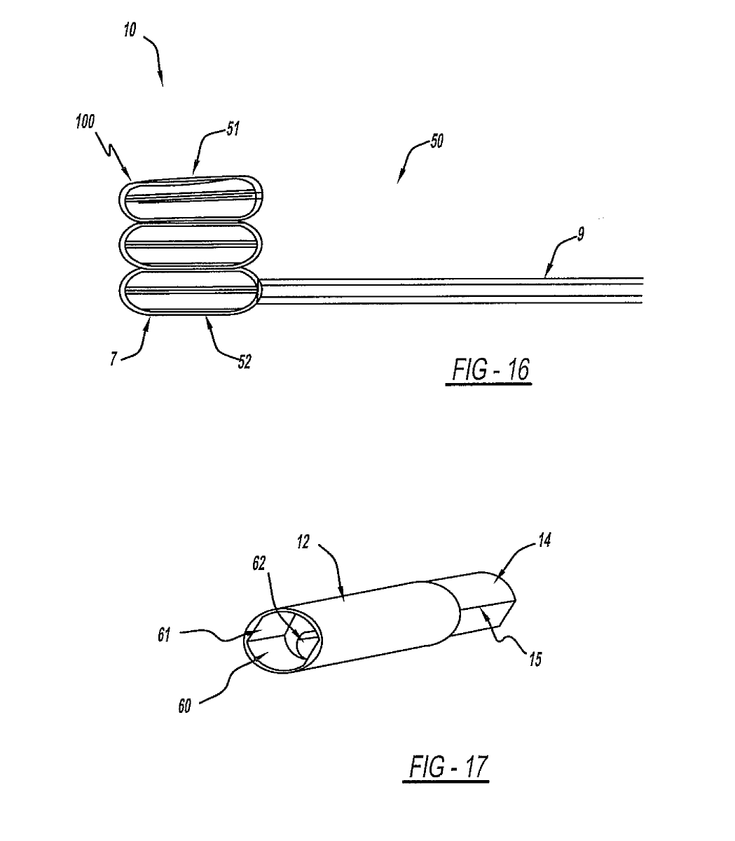

In FIG. 15, internal passages 53 allow for easy movement of the material, fluid, or gas through a single tube 9 to all of the partial rings forming the layers 104 of the device 50. The advantages of this variation are straightforward. First, the device acts as a powerful jack to push the end plates apart. Secondly, the large surface area of the upper surface 51 and lower surface 52 of the body 1 allow for better distribution of the correction loads created by expansion of the device and more accurate vertebral body restoration. Third, the device is temporary and does not stay in situ long term within the body. In addition, the removal of the additional tube and material for a hollow core design allows for a significant reduction of the overall collapsed packaged height and size, which makes it possible to insert the solid core device 50 down a smaller cannula. This is highly beneficial in the cervical spine or in cases where access to a vertebral body is limited or compromised. Of course, as in the case of the hollow core design 10, the upper surface of the device can be angled to aid in restoring Iodosis. Such a configuration is shown in FIG. 16. In fact, the upper or lower face can be angled, as shown in FIG. 16, such that surfaces 51 or 52 could be angled. Of course, both faces can be angled depending on the requirements of the circumstances of the surgery.

As stated above, it is possible to expand the solid core device with the hardenable fluid material. In this embodiment, a rigid implant is formed after the material hardens. Yet another variation is to adapt the benefits of the hollow core device and porous or semi-porous end caps discussed above and adapt them to the solid core device. Small openings in the solid core device, either on the upper or lower faces or both, or at numerous points along the sides of the device, allow both cement or an alternative hardenable material to expand the device and then exit in a limited, controlled fashion, through predetermined sized openings in the solid core 50. By adjusting the size of the openings relative to the viscosity of the material used to expand the solid core, restoration of the vertebral body height and geometry can be established while allowing controlled interdigitation and integration of the bone cement or other hardenable fluid with the vertebral body end plates and cancellous bone.

The above embodiment also opens up an opportunity to use different materials for the body of the device. In general, a polymer such as polyethylene or polyurethane or other flexible plastic can be used to create the flexible walls of the device 10, 50 for restoration of the vertebral body height. However, woven materials can be used which would be an advantage in creating a bioresorbable flexible device or for creating the pores or openings that allow controlled leakage of bone cement from the body 1 of the device as described above.

For insertion into the vertebral body by way of an opening in the pedicle or through the vertebral body, an instrument is used to hold the device, as briefly discussed above. This can be used through an open procedure or through a small percutaneous incision. FIG. 17 shows an embodiment of a cannula system, as briefly discussed above, whereby an external tube 12 is disposed over an internal rod or tube 14 machined or formed to have a sufficient opening 62 to allow the device tubes 8 and 9 to pass through the instrument. The external tube or cannula 12 is keyed to the internal tube 14 via a keyway or flat 61 on the inside of the external tube and a matching feature or flat 15 such that the correct orientation of the device can be determined after insertion of the device into the vertebrae, as discussed in detail above. The end of the internal tube 14 is set back from the end of the external tube 12 to create an open space inside of the cannula 15 at its tip. The device 10 is held in the opened space of the cannula during insertion and until deployment.

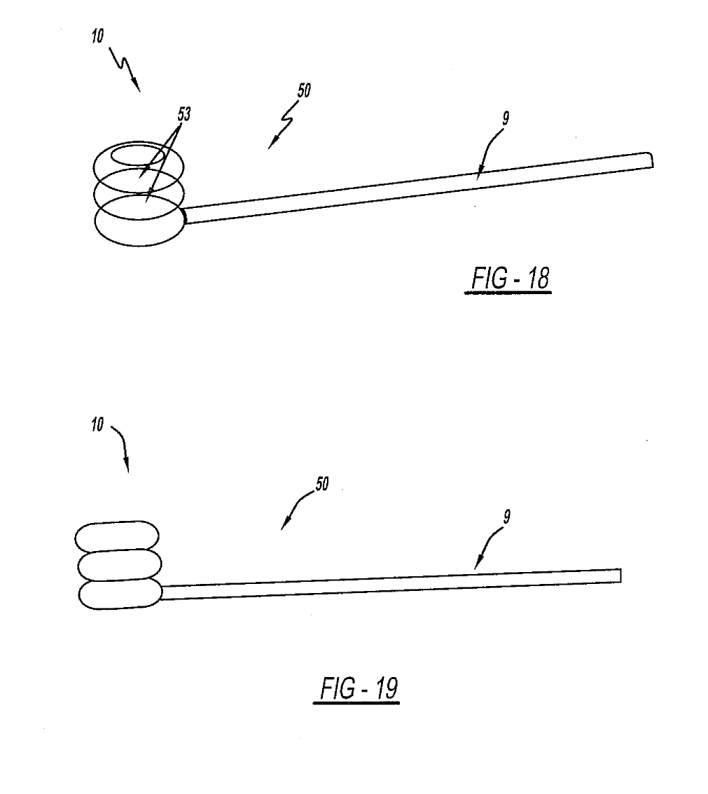

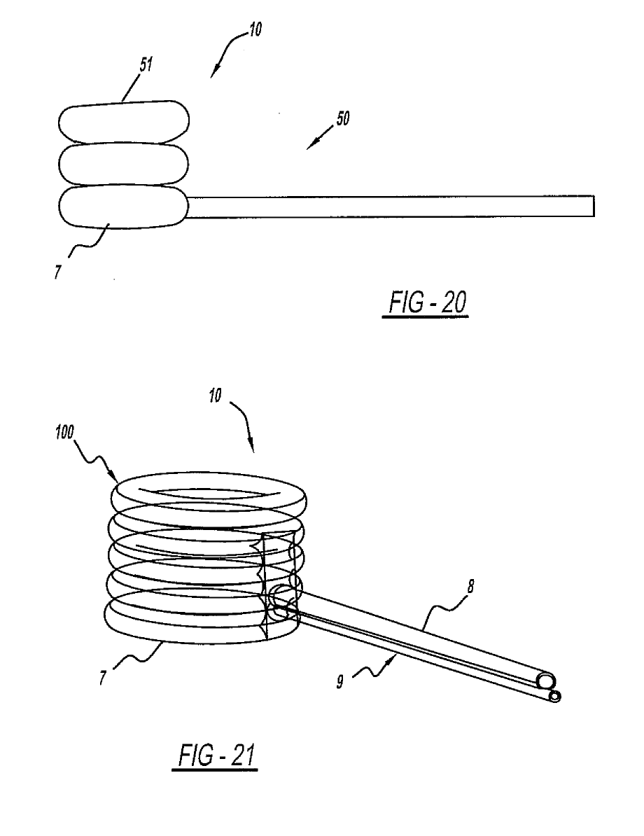

FIGS. 18-24 provide shaded images of the variations discussed above to better show the devices 10, 30, 50. FIG. 18 shows the solid core device 50 whereby internal open passages 53 are readily seen. FIG. 19 is a side view of the solid core device. FIG. 20 is a shaded image of the solid core device whereby the upper surface 51 is angled relative to the lower surface 7. Either or both the upper and lower surfaces can be angled, or the angled face or faces can be in the opposite directions for reasons discussed above.

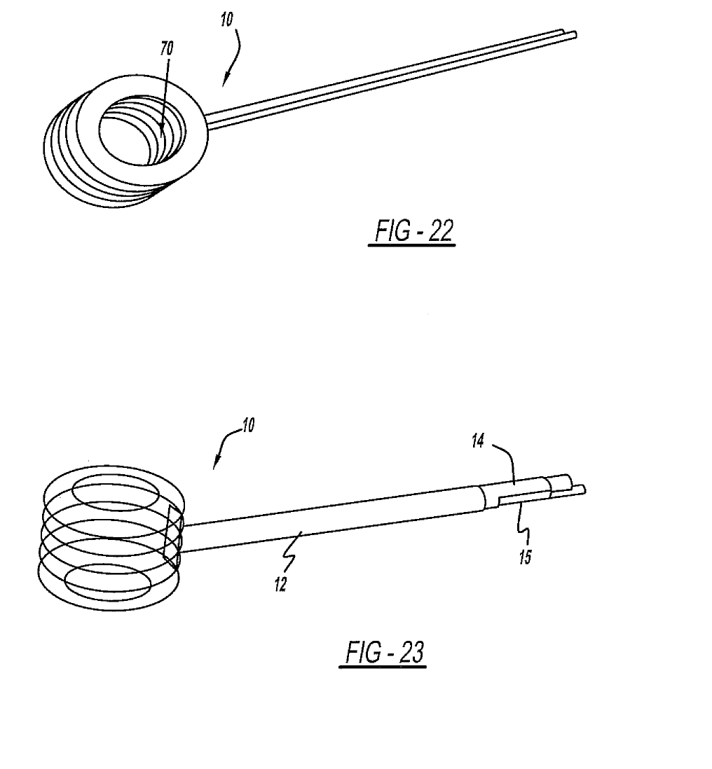

FIG. 21 is a transparent rear perspective view showing the various tubular rings of the hollow core device 10, the reinforcement and rear manifold 20, and the filler tubes 8 and 9. FIG. 22 provides a view of the bone cement and hardening material injection tube opening 70 into the center of the hollow core device 10.

FIG. 23 provides a transparent view showing the cannula system 12 with the hollow core device 10. The internal tube 14 also projects and provides support to tubes 8 and 9 during the inflation/enlargement and injection processes.

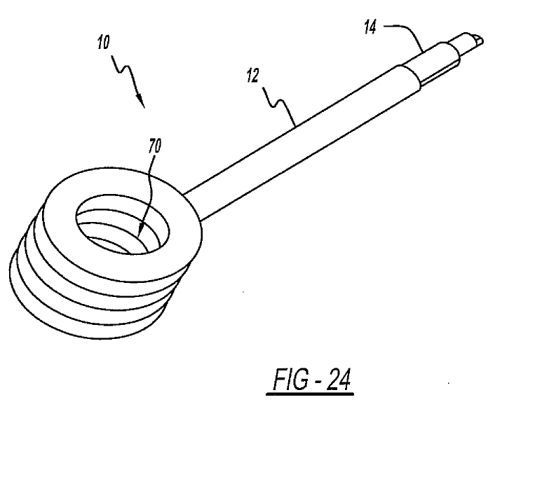

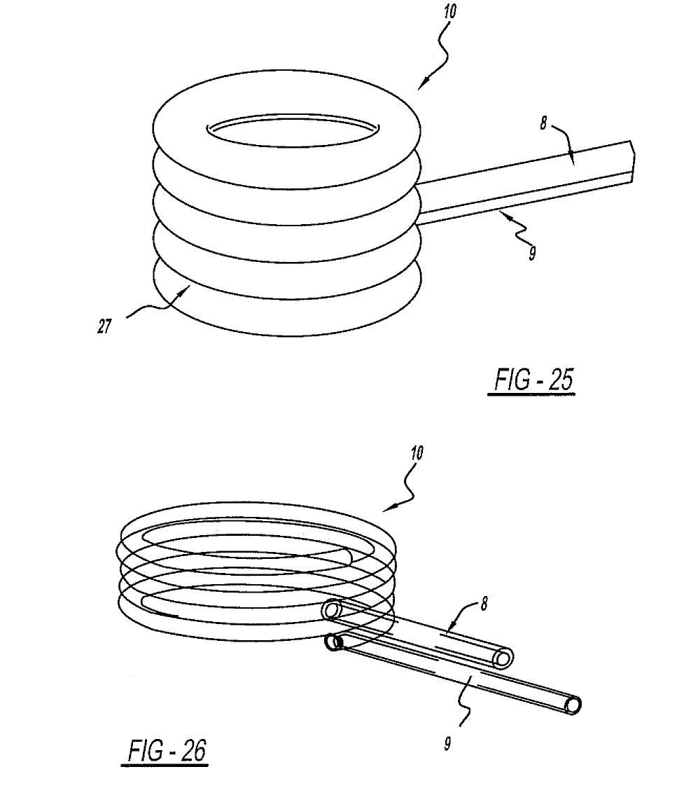

FIG. 24 provides an additional view of the embodiment in FIG. 23, whereby the tip 70 of the injection tube 8 is visible. FIG. 25 is an enlarged view which also shows openings for allowing fluid or gas to move from chamber to chamber as previously described.

FIG. 26 shows an alternative construction of the present invention in the form of the hollow core design 10. Rather than having the chambers formed of rows or layers of individual chambers, the chambers are formed in a helical fashion such that the tube is wound as if in a spring form. The tubes can float in a stack or be interconnected such that the wall of one tube is fixed to at least one other tube. This creates a hollow core device with a simpler internal passageway (a single internal passageway) for expansion with fluid injected thereinto through tube 8.

There are numerous methods of manufacturing the present invention and various variations thereof which such as by molding or other forming techniques. Injection molding around a core, which is removed after the injection process is complete, is a standard method of molding flexible parts. An alternative is that the individual chambers can be formed and bonded via plastic or solvent welding, or utilizing adhesives, along with the fluid and bone cement tubes. An alternative way of manufacturing the device 10 is by utilizing a tube of flexible material that is rolled over such that a section of the tube slides over the other sections which then become inside the other tube. This is simply a way of making a tube within a tube from one piece of tubing. The chambers are then heat sealed and formed and the feed tubes are attached by heat sealing, welding, or by other adhesives known in the art.

In view of the above, the present invention provides a novel method of restoring height to a collapsed intra-vertebral space by inserting a body 1 into the intra-vertebral space defined by opposing intra-vertebral surfaces and selectively and reversibly expanding layers 104 of the body 1 causing top and bottom surfaces 100, 102 of the body 1 to contact and separate the opposing vertebral surfaces thereby expanding the intra-vertebral space. More specifically, fluid is supplied through the fluid inlet tube 9 to an inner chamber of the body 1 to expand the layers 104 of the body 1. In one embodiment, the layers are expanded around a hollow central core 108 of the body 1 and then a hardenable fluid is delivered to the hollow core 108. Preferably, the hardenable fluid is delivered to all of the layers through a single fluid inlet 9. Once the hardenable fluid is allowed to harden, the body 1 is collapsed and removed from the intra-vertebral space.

As discussed above, the inventive method further allows for the flowing of hardenable material out of the ends of the hollow core 108 to contact adjacent opposing vertebral surfaces. This process can also be accomplished by injecting the hardenable material into a body without a central core, utilizing the hardenable material to expand the body. The process can include the further step of allowing leakage of the hardenable material from the solid core embodiment for the purposes described above.

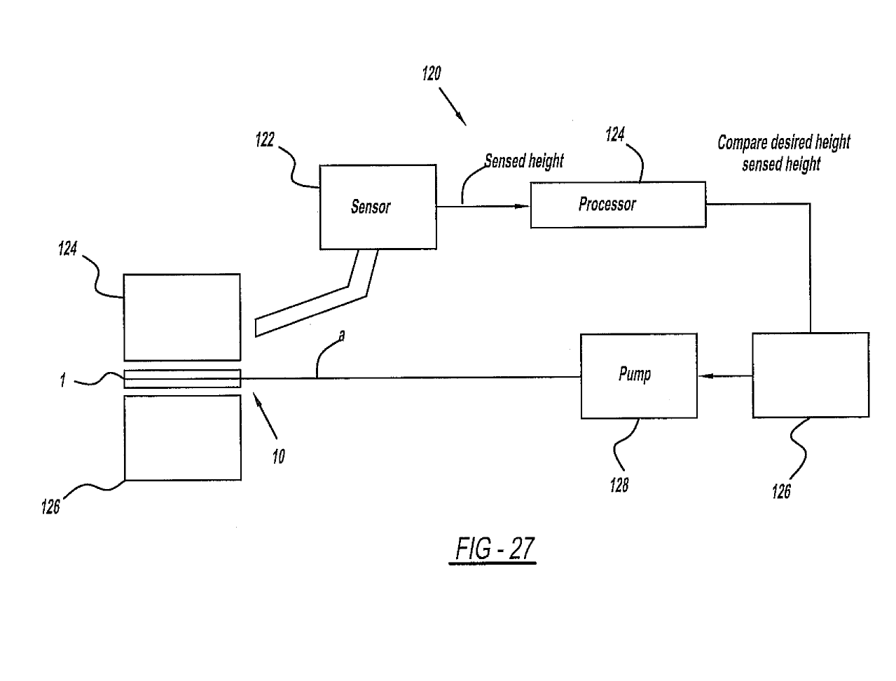

An automated control system for automatically expanding and collapsing the body 1 of the device 10 is shown generally at 120 in FIG. 27. The automated system provides a programmable control mechanism for controlling expansion of the body 1 to a predetermined height to a pre-selected height.

More specifically, the system 120 includes a sensor 122 for sensing the height of the collapsed intra-vertebral space defined by the space between the two vertebrae shown in FIG. 27, schematically show at 124 and 126. The sensor could be a visual imager capable of translating a visual image into digital information, such as a MRI, CAT, or other visual imaging device. The sensed height is then delivered to a processor 124 which compares the sensed height to a predetermined desired height. This desired height could be programmed by the physician after inspection of the collapsed intra-vertebral stays or could be pre-programmed based on population data. The processor 124 utilizes the comparison to actuate a feedback control system 126 which controls pump 128 to continue to feed fluid through tube 9 for expanding body 1. This feedback loop controls the automatic feed of fluid into the body 1 thereby automatically expanding body 1 to a predetermined size or shape. What is critical is the expansion of the intra-vertebral space to a predetermined height. This can be sensed either by back pressure through the pump into the feedback control or visually through the sensor 122 providing data to the processor which performs the comparing function.

In view of the above, the present invention provides various advantages over the prior art. The present invention provides a multichamber device that can be inserted into a small opening and then expanded to a larger size. Upon expansion, a broad surface is created to contact areas for aiding and pushing the vertebral end plates back to the proper anatomical position. Simply, all chambers can be expanded through a single tube. Alternatively, at least one of the chambers can be separately expanded through a second tube. In other words, either manually or through an automated system, various layers of the body 1 can be individually expanded depending upon the size and shape needed to properly contact and separate the vertebral surfaces. The present invention further provides means for correcting lordosis by various methods and at various angles. The present invention further provides novel means for allowing controlled release of hardenable material through the device in a selective and controlled manner. Finally, the present invention provides a novel automated system allowing for precise expansion of the vertebral space to a desired height.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. An intra-vertebral body height restoring device comprising:
a body for insertion into an intra-vertebral space and including top and bottom surfaces for engaging opposing vertebral surfaces defining the intra-vertebral space, said body including at least two layers extending along a width of said body and having a fully expanded and fully collapsed height relative thereto; and
reversible expansion means for selectively and reversibly expanding and collapsing the height of said layers between and including said fully expanded and collapsed heights to restore a selected height of the intra-vertebral space.

2. The device as set forth in claim 1, wherein said body includes a chamber defining said layers.

3. The device as set forth in claim 2, wherein said body includes a helically spiraling inner surface defining said chamber.

4. The device as set forth in claim 3, including fluid inlet means for selectively and reversibly supplying a fluid to said inner chamber and expand or collapse said body.

5. The device as set forth in claim 4, wherein said inlet means includes a supply tube in fluid communication with said inner chambers.

6. The device as set forth in claim 5, wherein said body includes an inner wall defining a open space therein for receiving a hardenable fluid therein.

7. The device as set forth in claim 6, including a second fluid inlet in fluid communication with said open space for delivering the hardenable fluid to said open space.

8. The device as set forth in claim 7, including dual filling means including said first and second fluid inlets for providing a single tube assembly for filling both said inner chamber and said open space.

9. The device as set forth in claim 8, wherein said dual filling means includes a second tube in fluid communication with said open space molded inside of said first mentioned tube which is in fluid communication with said inner chamber.

10. The device as set forth in claim 7, wherein said body further includes manifold means for allowing fluid flow between said layers.

11. The device as set forth in claim 10, wherein said manifold means includes a web of strengthening material extending through said layers for strengthening a connector between said fluid inlet means and said body.

12. The device as set forth in claim 6, wherein said open space opens into said top and bottom surfaces defining a body around a hollow core.

13. The device as set forth in claim 12, wherein said body includes end caps seal over said hollow core proximate to said top and bottom surfaces for containing the hardenable material injected into said hollow core.

14. The device as set forth in claim 13, wherein said end caps are porous for allowing selective leakage of the hardenable material injected into said hollow core, from said hollow core and into adjacent vertebral surfaces.

15. The device as set forth in claim 1, including lordosis restoring means for restoring proper lordosis to a spine.

16. The device as set forth in claim 15, wherein said layer adjacent one of said top and bottom surfaces is angled relative to said remaining layers defining said lordosis restoring means.

17. The device as set forth in claim 15, wherein both of said top and bottom surfaces are angled relative to said remaining layers defining said lordosis restoring means.

18. The device as set forth in claim 2, wherein said body includes porous top and bottom surfaces for containing a hardenable fluid in said inner chamber while allowing selective leakage of hardenable fluid through said top and bottom surfaces into the opposing vertebral surfaces.

19. The device as set forth in claim 3, wherein said body includes a single tube member, said tube member being a helical spiral defining said layers.

20. The device as set forth in claim 19, including at least top and bottom layers each of said layers including top and bottom surfaces, said top and bottom surfaces being operatively connected to adjacent top and bottom surfaces.

21. The device as set forth in claim 2, wherein said body includes a plurality of stacked independent chambers defining said at least two layers.

22. The device as set forth in claim 21, including inlet means operatively connected to each of said chambers for selectively expanding and/or collapsing each of said chambers independently of each other.

23. The device as set forth in claim 22, wherein said inlet means includes a supply tube in fluid communication with said inner chambers.

24. The device as set forth in claim 23, wherein said body includes an inner wall defining a open space therein for receiving a hardenable fluid therein.

25. The device as set forth in claim 24, including a second fluid inlet in fluid communication with said open space for delivering the hardenable fluid to said open space.

26. The device as set forth in claim 25, including dual filling means including said first and second fluid inlets for providing a single tube assembly for filling both said inner chamber and said open space.

27. The device as set forth in claim 26, wherein said dual filling means includes a second tube in fluid communication with said open space molded inside of said first mentioned tube which is in fluid communication with said inner chamber.

28. The device as set forth in claim 25, wherein said body further includes manifold means for allowing fluid flow between said layers.

29. The device as set forth in claim 26, wherein said manifold means includes a web of strengthening material extending through said layers for strengthening a connector between said fluid inlet means and said body.

30. The device as set forth in claim 24, wherein said open space opens into said top and bottom surfaces defining a body around a hollow core.

31. The device as set forth in claim 30, wherein said body includes end caps seal over said hollow core proximate to said top and bottom surfaces for containing the hardenable material injected into said hollow core.

32. The device as set forth in claim 31, wherein said end caps are porous for allowing selective leakage of the hardenable material injected into said hollow core, from said hollow core and into adjacent vertebral surfaces.

33. An intra-vertebral body height restoring device comprising:
a body defining a width and height and including an inner portion defining at least two layers extending along a width of said body; and
expansion means for selectively and reversibly expanding said height of said layers.

34. An intra-vertebral body height restoring device comprising:
a body;
reversible expansion means for selectively and reversibly expanding and collapsing said body; and
containment means within said body for containing a hardenable fluid therein.

35. An inter-vertebral body height restoring device comprising:
a body;
containment means within said body for containing a hardenable fluid therein and;
porous surface means for allowing a selective amount of flow of the hardenable material from said containing means out through at least one surface of said body for contact with a vertebral surface adjacent to said one surface.

36. A method of restoring height to a collapsed intra-vertebral space by:
inserting a body into the intra-vertebral space defined by opposing intra-vertebral surfaces; and
selectively and reversibly expanding layers of the body causing top and bottom surfaces of the body to contract and separate the opposing vertebral surfaces thereby expanding the intra-vertebral space.

37. The method as set forth in claim 36, wherein said expanding step is further defined as supplying fluid through a fluid inlet to an inner chamber of the body to expand the layers of the body.

38. The method as set forth in claim 37, wherein said supplying step is further defined as delivering fluid simultaneously to all layers of the body from a single fluid inlet.

39. The method as set forth in claim 37 further including the step of expanding the layers of the body around a hollow central core of the body and then delivering a hardenable fluid to the hollow central core.

40. The method as set forth in claim 39, including a further step of collapsing the body after the delivered hardenable fluid has become hard and then removing the body from the intra-vertebral space.

41. The method as set forth in claim 39, further including a step of flowing the hardenable fluid out of ends of the hollow central core to contact adjacent opposing vertebral surfaces.

42. The method as set forth in claim 41, wherein said flowing step is further defined as containing the hardenable fluid within the hollow central core and allowing selective leakage of the contained hardenable fluid onto the adjacent vertebral surfaces.

43. The method as set forth in claim 36, including the further step of restoring lordosis to the spine.

44. The method as set forth in claim 43, wherein said restoring step is further defined as expanding an angled top and/or bottom layer of the body to apply an angulated pressure and the adjacent vertebral surfaces.

45. A method of restoring height to a collapsed intra-vertebral space by:
expanding a body disposed within the intra-vertebral space to separate opposing vertebral surfaces defining the space;
injecting bone cement into the expanded body; and
containing the bone cement within the body.

46. A method of restoring height to a collapsed intra-vertebral space by:
injecting a hardenable material into layers of a body;
expanding the height of the body with the hardenable material to separate adjacent vertebral surfaces defining the intra-vertebral space; and
hardening the hardenable material to fixedly space the vertebral surface.

47. A method of restoring height to an intra-vertebral space by:
expanding a body containing a hardenable fluid within the intra-vertebral space to separate opposing vertebral surfaces defining the space; and
selectively leaking the hardenable fluid through permeable??? top and bottom surfaces of the body to contact the hardenable fluid with selected portions of the adjacent vertebral surfaces.

48. A method of restoring height to a collapsed intra-vertebral space by:
inserting a cannula containing an oriented collapsed body into an intra-vertebral space;
ejecting the oriented collapsed body from the cannula and into the intra-vertebral space with the top and bottom surfaces of layers of the body being oriented to face opposing vertebral surfaces defining the intra-vertebral space; and
expanding the layers of the body to engage the top and bottom surfaces with the opening vertebral surfaces to expand the height of the intra-vertebral space.

49. A device for restoring the height of a collapsed intra-vertebral space comprising:
an expandable body; and
programmable control means for controlling expansion of said body to a predetermined height in view of a pre-selected height.

50. The device as set forth in claim 49, wherein said programmable control means includes sensing means for sensing the height of the collapsed intra-vertebral space; and
feedback means for controlling expansion of said body and stopping expansion of said body when said body restored the height of the inter-vertebral space to said predetermined height.

51. The device as set forth in claim 50, further including fluid inlet means for supplying fluid to an inner chamber of said body to expand said body, said feedback means controlling the supply of fluid through said fluid inlet means based on information from said sensing means.

52. The device as set forth in claim 51, further including:
pumping means for injecting the hardenable fluid into said body operatively connected to said fluid inlet means and said feedback means; and
processor means operatively connected between said sensor means and said feedback means for comparing sensed height from said sensor means with a predetermined height and actuating said feedback means to actuate said pressuring means to increase flow of hardenable fluid into said body to increase the sensed height to said desired height.

53. An intra-vertebral body height restoring device comprising:
at least two enlargable chambers having an expanded condition and collapsed condition.
